(19) United States
(12) Patent Application Publication
(10) Pub. No.: US 2008/0214913 A1
(43) Pub. Date: Sep. 4, 2008

(54) Surface-Enhanced Spectroscopy with Implanted Biosensors

(75) Inventors: Antonius Theodorus, Martinus Van Gogh, Eindhoven (NL); Gert 'T Hooft, Eindhoven (NL); Gerhardus Wilhelmus Lucassen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/816,534

(22) PCT Filed: Feb. 16, 2006
(86) PCT No.: PCT/IB06/50510

§ 371 Date: Aug. 17, 2007

(30) Foreign Application Priority Data
Feb. 22, 2005 (EP) 05101329.0

(51) Int. Cl. A61B 5/1455; A61B 5/00

(52) U.S. Cl. 600/318; 600/316; 600/322

(57) ABSTRACT

The present invention provides a spectroscopic apparatus, a method and a computer program product for determining the concentration of an analyte of a fluid that flows through a capillary vessel of a biological sample. The spectroscopic apparatus makes use of an imaging system for determining the position of at least one biosensing substrate that has been implanted into the biological sample in the proximity of the capillary vessel but outside the capillary vessel. The biosensing substrate is capable of inducing surface-enhanced spectroscopic effects and is preferably adapted to reversibly and selectively bind a certain analyte or molecule of the fluid, to which the vessel wall of the capillary vessel is at least semi-permeable. By performing a spectroscopic analysis not directly inside the capillary vessel but in close proximity to the capillary vessel, the signal-to-noise ratio of the spectroscopic signals obtained can be appreciably enhanced while disadvantageous scattering and interference effects can be minimized.

The present invention relates to the field of spectroscopy, and more particularly without limitation, to Surface-Enhanced Raman Spectroscopy (SERS) that is applicable to biological samples.

Within the framework of spectroscopy and in particular in Raman Spectroscopy, surface-enhanced effects leading to Surface-Enhanced Raman Spectroscopy (SERS) provide a highly accurate and sensitive detection of analytes and substances of a sample. Generally, a rather weak signal arising from a Raman scattering process can be greatly strengthened if the molecules that are subject to Raman scattering are attached or positioned close to nanometer-size noble metal structures. When a scattering molecule is in close proximity to a metal surface, an enhancement in Raman intensity arises from coherent superposition of the incident and reflected fields at the position of the molecule and due to excitation of surface plasmons by electromagnetic radiation. The means for inducing the surface-enchanced spectroscopic effect are preferably designed for Surface-Enhanced Raman Spectroscopy. Hence, SERS has the potential to increase the Raman signal by up to 8 to 12 orders of magnitude. Therefore, by means of SERS even detection of single molecules within a sample might become applicable.

A wide application range for SERS is in medical diagnostics. In principle, SERS can be utilized for determining concentrations of certain analytes that are disolved in bodily fluids. In this way, for example a glucose concentration of blood flowing through the vascular system of a person or an animal can be precisely determined in vivo in a minimal invasive way. However, for the purpose of a blood glucose concentration measurement the nano particles inducing the surface-enhanced effect have to be injected into the blood stream or into a blood vessel in order to obtain the desired surface-enhanced spectroscopic effect.

U.S. patent application US 2004/0180379 A1 discloses Surface-Enhanced Raman nanobiosensors for detection of analytes. These nanobiosensors might be realized as EG3-modified silver film over nanosphere surfaces (AgFON). Such nanobiosensors provide reversible binding. Also, a dialysis membrane can be utilized to exclude molecules that are significantly larger than the analyte of interest, e.g. glucose, from contacting the nano-particle surface. Hence, an EG3-modified AgFON surface provides reversible and selective binding of an analyte for applications in the framework of Surface-Enhanced Raman Spectroscopy.

However, making use of nanobiosensors injected into a bodily fluid for SERS purposes, various disadvantages have to be taken into account. Performing Surface-Enhanced Raman Spectroscopy directly in a blood stream or in a blood vessel, the efficiency of the spectroscopic effect is severely affected by scattering processes with red blood cells. Furthermore, due to the aspect that there exist many different analytes in the blood, the obtained Raman signal inherently represents spectroscopic information of many constituents of the blood. Additionally, the plurality of different Raman signals might be subject to interference, which further complicates the detection of a particular analyte or constituent of the bodily fluid, such as blood.

Further, by performing SERS the signal strength strongly depends on the morphology of the injected nano-sized particles. Consequently, for a constant signal strength it has to be provided that either the surface of the substrate is rather homogeneous or that the spectroscopic excitation radiation is always focused on the same spot on the surface.

The present invention therefore aims to provide an apparatus and a method as well as a computer program product for improving accuracy, reliability as well as patient comfort of minimal invasive in vivo concentration determination of analytes in bodily fluids.

The present invention provides a spectroscopic apparatus that is adapted to determine a concentration of an analyte of a fluid located in a volume of a biological sample, that is confined by walls that are at least semi-permeable for the analyte of the fluid. An example of such a volume is a capillary vessel, with the capillary vessel wall representing an at least semi-permeable membrane. The inventive apparatus comprises an imaging system for determining a position of at least one substrate that has been implanted into the biological sample in the proximity of the volume. Here, the substrate is not implanted within but outside the volume, i.e the capillary vessel, typically in close proximity to the vessel walls. This at least one substrate is further capable of inducing a surface-enhanced spectroscopic effect, like a Surface-Enhanced Raman Spectroscopic effect.

The spectroscopic apparatus further comprises a light source for generating excitation radiation and a focusing arrangement for focusing of the excitation radiation onto the implanted substrate. Further, the spectroscopic apparatus comprises a radiation detector for detecting return radiation returning from the biological sample and a spectroscopic analysis system for spectrally analyzing the detected return radiation and for determining the concentration of the analyte by making use of the detected return radiation.

For maximum benefit of the spectroscopic apparatus, it is required that the at least one substrate be implanted into the biological sample in the proximity of the volume that contains the bodily fluid in which the analyte of interest is typically dissolved. Since the wall is semi-permeable for the analyte of interest, the analyte may penetrate through the wall and may become subject to a physiological transport mechanism, such as diffusion, in the biological sample or in the tissue. For example by placing the surface-enhancing substrate next to a capillary vessel, only those analytes of the fluid that are capable of penetrating through the vessel wall can become subject to a surface-enhanced spectroscopic effect induced by the implanted substrate. In this way other substances whose atoms, ions, molecules or clusters of molecules that are not capable of penetrating through the vessel wall and may spoil the spectroscopic effect cannot become subject to Surface-Enhanced Raman Spectroscopy.

Typically, the substrates are implanted in a well-defined way with respect to the location and position of the volume. Preferably, the at least one substrate is implanted in close proximity to the volume in order to guarantee that the concentration of the analyte of interest does not become too low due to diffusion in the surrounding tissue of the volume. In essence, the entire spectroscopic procedure is no longer performed in the volume itself, e.g., the capillary vessel, but makes effective use of the fact that the analyte of interest, whose concentration has to be determined by means of the spectroscopic analysis, is capable of penetrating through the vessel wall. In this way scattering effects of e.g. red blood cells as well as interference of Raman signals of various constituents can be reduced to a minimum. This provides an improved sensitivity and specificity and hence an increased signal-to-noise ratio and, consequently, provides an improved accuracy of the entire spectroscopic analysis.

Further, the signal enhancing substrates are typically injected at a location close to the capillary vessel wall in order to keep the time delay between the physiologically relevant analyte concentration in the capillary vessel and the local analyte concentration at a low level. The distance between the location and the capillary vessel wall is typically governed by the diffusion constant of the analyte in the surrounding tissue and is chosen such that the change in concentration of the analyte in the capillary vessel can be detected within a predetermined time interval. This time interval should be typically negligible with respect to the time scale of physiologically relevant concentration variations.

According to a further embodiment, the focusing arrangement is controllable by means of an output of the imaging system for selectively focusing of the excitation radiation on the implanted substrate. Hence, the spectroscopic apparatus comprises a feedback mechanism for an autonomous detection of implanted substrates in order to selectively focus the spectroscopic excitation radiation onto the implanted substrate. Hence, the imaging system is provided with pattern recognition means in order to recognize implanted substrates in the biological sample. Once an implanted or several implanted substrates have been identified and recognized by the imaging system, the imaging system may further perform an optimization procedure in order to select at least one of the implanted substrates that will become subject to irradiation with excitation radiation. In this way the spectroscopic apparatus features an autonomous substrate recognition and identification for selective focusing of excitation radiation on the implanted substrates.

Further, the imaging system may derive a distance parameter indicating the distance between the capillary vessel and the implanted substrate that becomes subject to focusing of excitation radiation. By means of such a distance parameter a correlation between a determined concentration of the analyte at the location of the substrate and the concentration of the analyte in the fluid that is located inside the capillary vessel can be derived. This allows to determine precisely the concentration of the analyte of the fluid by measuring the concentration of the analyte not in the fluid but at a determined distance outside a capillary vessel. The distance parameter may further allow to determine a delay and hence a time correlation between the concentration changes of the analyte in the capillary vessel with respect to the location outside the capillary vessel.

Moreover, the present invention allows to determine an absolute position of a detected implanted substrate which facilitates a calibration of the spectroscopic system and a repeated focusing on the implanted substrate for a successive spectroscopic analysis.

In a further embodiment the imaging system is adapted to determine the position of the at least one implanted substrate by means of a detectable mark on the at least one implanted substrate. In this embodiment the implanted substrates features for instance a specific fluorescent mark providing a fluorescent optical signal in response to respective illumination. In this way identification and allocation of implanted substrates within the bulk of the biological sample can be facilitated. Also, by making use of specific fluorescent marks reliability and effectivity of substrate identification and substrate recognition can be effectively enhanced.

In a further embodiment, the spectroscopic apparatus is applicable to determine the concentration of an analyte of blood that is flowing through the vascular system of a person or an animal. Here, the analyte is able to penetrate through the vessel walls of the vascular system of the person or animal. Also, the spectroscopic apparatus is applicable to blood vessels in general and does not require a flow of blood. Typically, the spectroscopic apparatus is applicable to determining a concentration of glucose, fatty acids, such as cholesterol, hormones or even proteins and vitamins. Consequently, the spectroscopic apparatus provides a non-invasive and in vivo determination of e.g. glucose concentration in blood, provided that the substrate has already been implanted subcutaneously into the respective body part of the person or animal. Hence, application of the inventive concentration determination only requires a minimally invasive procedure for the implantation of the surface-enhancing substrates. Once the surface-enhancing substrates have been implanted into the biological tissue, the inventive spectroscopic apparatus can be repeatedly applied in a non-invasive way for concentration determination of various analytes leading to a substantial increase of patient comfort.

In another embodiment the spectroscopic apparatus is applicable to the cornea of a person or animal and is further adapted to determine a concentration of an analyte of the aqueous humour. Also here application of the inventive concentration determination requires a minimally invasive step of implanting or injecting surface-enhanced substrates into the cornea or into the aqueous humour of an eye. However, once being implanted, the surface-enhancing substrates can be repeatedly applied for various subsequent procedures to determine the concentration.

Also, the invention can be applied to tear fluid of the eye in combination with signal enhancing substrates that are applied to the outer surface of the cornea, e.g., by a specially prepared contact lens. In such an embodiment the inventive method would even be non-invasive.

In a further preferred embodiment, the at least one substrate comprises a noble metal and is further adapted to adsorb the analyte. Hence, the at least one substrate has to fulfill the general requirements of a nano-particle that is capable of inducing a surface-enhanced spectroscopic effect, such as SERS.

In a further embodiment, the at least one substrate is further adapted for reversible and selective adsorption of the analyte. Hence, once adsorbed to the substrate, the analyte is not permanently bound or attached to the surface-enhancing substrate. In this way the amount of adsorbed analytes corresponds to the concentration of the analyte in the surrounding tissue or in the surrounding bodily fluid.

Further, the surface-enhancing substrate provides selective adsorption of the analyte. For instance, the at least one substrate is designed for adsorption of glucose molecules but does not provide adhesion of large proteins, like albumin. In this way only dedicated analytes or molecules can adhere to the at least one substrate and consequently the derived spectroscopic signal is exclusively representative of the concentration of this specific molecule, which leads to an increased signal-to-noise ratio.

In a further embodiment, the substrate comprises a monolayer of noble metal that features a thickness in the nanometer range and/or wherein the substrate further comprises noble metal nano-particles. For instance, the substrate might be realized by means of an EG3-modified AgFON surface. Such dedicated biosensors provide selective and reversible, i.e. non-permanent, adsorption of an analyte of interest, such as glucose.

In another aspect, the invention provides a method of determining a concentration of an analyte of a fluid that is flowing through a capillary vessel of a biological sample. The capillary vessel is at least semi-permeable for the analyte of the fluid and the method comprises the steps of determining a position of at least one substrate that has been implanted into the biological sample in the proximity of the capillary vessel outside the capillary vessel. This at least one substrate is capable of inducing a surface-enhanced spectroscopic effect. The method further comprises the step of focusing excitation radiation on the implanted substrate and detecting return radiation emanating from molecules or analytes that are bound to the implanted substrate. Further the method comprises the step of spectrally analyzing the detected return radiation for determination of the concentration of the analyte.

In a further embodiment the method is applicable to in vivo determination of the concentration of an analyte of blood that is flowing throught the capillary vessel. Here, the biological sample comprises a human or animal body part and the analyte is able to penetrate through the vessel wall of the vascular system of the respective body part. In typical implementations, the method is applicable to the determination of the concentration of glucose of blood.

In a further preferred embodiment of the invention, the method is applicable to the cornea of a person or an animal and is further adapted to determine a concentration of an analyte of the aqueous humour of the eye of the person or the animal.

In another aspect, the invention provides a computer program product for a spectroscopic apparatus that is adapted to determine a concentration of an analyte of a fluid that is flowing through a capillary vessel of a biological sample. The capillary vessel is at least semi-permeable for the analyte of the fluid and the computer program product comprises computer program means that are operable to process an output signal of an imaging system to determine a position of at least one substrate that has been implanted into the biological sample in the proximity of the capillary vessel outside the capillary vessel. This at least one implanted substrate is capable of inducing a surface-enhanced spectroscopic effect. The computer program means are further operable to control a focusing arrangement for focusing of excitation radiation onto the implanted and recognized substrate and is further operable to process an output signal of a radiation detector for spectral analysis of the detected return radiation. Processing of the output signal of the radiation detector provides determination of a concentration of the analyte, since the output signal is indicative of return radiation detected by means of the radiation detector.

In the following preferred embodiments of the invention will be described in detail by making reference to the drawings in which:

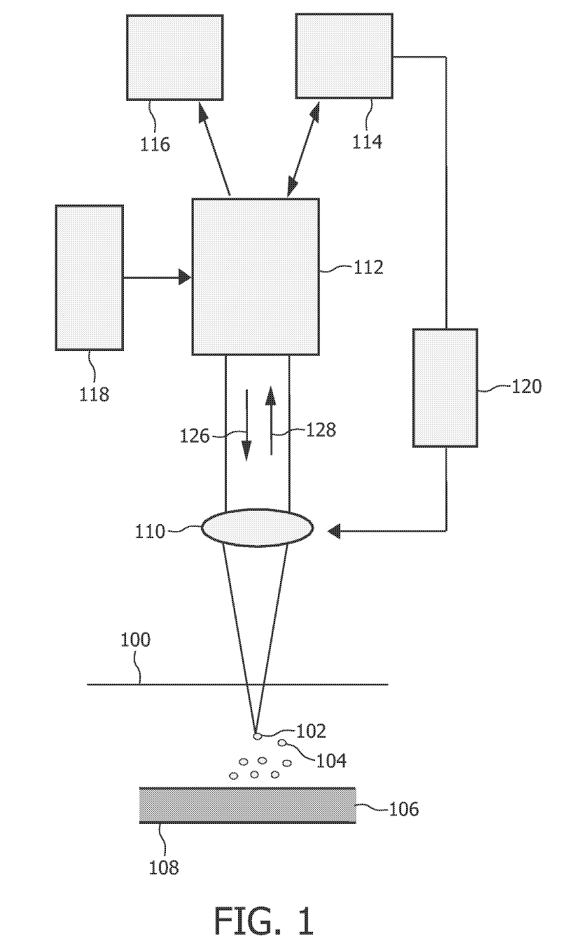

FIG. 1 schematically illustrates a block diagram of the spectroscopic apparatus,

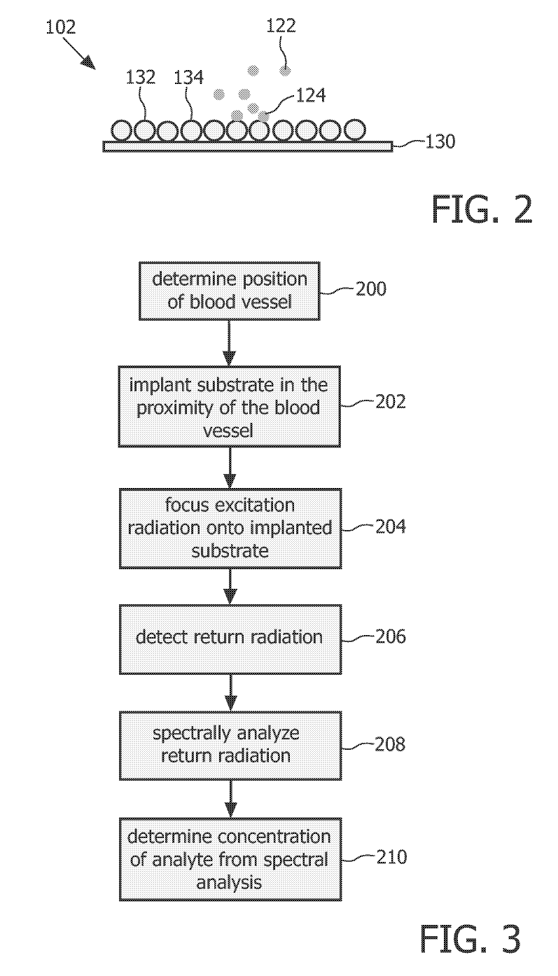

FIG. 2 illustrates a cross section of the biosensing substrate,

FIG. 3 shows a flowchart of performing the method of determining the analyte concentration.

FIG. 1 shows a schematic block diagram of the spectroscopic apparatus and its main components. The spectroscopic apparatus comprises an objective lens 110, a light source 118, a spectroscopic system 116, an imaging system 114, a light coupling arrangement 112 and an objective control 120. In the illustrated embodiment, the spectroscopic apparatus is applicable to skin tissue of e.g. a human patient. The tissue or the body part of the human patient comprises a blood vessel 106 underneath the surface of the skin 100. Further, various biosensing substrates 102, 104 have already been implanted in the bulk of the tissue in the proximity of the blood vessel 106. Hence, the biosensing substrates 102, 104 are implanted in close proximity to the vessel walls 108 of the blood vessel but outside the blood vessel itself.

The implanted biosensing substrates 102, 104 themselves are capable of selectively and reversibly binding of a dedicated analyte of the blood, such as e.g. glucose. Since the biosensing substrate typically comprises nanometer-size noble metal particles or noble metal spheres, adhesion or adsorption of analytes to the biosensing substrate provides a surface-enhanced spectroscopic effect, such as SERS when being subject to irradiation with spectroscopic excitation radiation, e.g. in the near infrared or infrared wavelength range. Adsorption of a dedicated analyte, like glucose to the biosensing substrates 102, 104 requires penetration of the analyte through the vessel walls 108. Therefore, the inventive spectroscopic apparatus and inventive method is only applicable to analytes for which the vessel walls 108 of the blood vessel 106 are permeable or at least semi-permeable.

Further, the biosensing substrates 102, 104 preferably provide reversible, i.e. non-permanent binding or adsorption of the dedicated analyte. In this way, the amount of molecules or analytes that are adsorbed by the substrates 102, 104 is a measure for the analyte concentration around a selected substrate 102. Due to the fact that the blood vessel wall 108 is at least semi-permeable to glucose, the number of glucose molecules adsorbed at substrate 102 is directly correlated to the glucose concentration of the blood flowing through the blood vessel 106. Hence, by making use of a Surface-Enhanced Raman Spectroscopic (SERS) effect taking place not inside but in the vicinity of the blood vessel 106 a precise determination of the analyte concentration can be performed.

Determination of analyte or glucose concentration by means of biosensing substrates in close proximity but outside the blood vessel 106 has the advantage of reducing the impact of scattering of excitation radiation at red blood cells and reducing the interference between Raman signals of various analytes.

The inventive spectroscopic apparatus and inventive method of determination of analyte concentration is analyte selective in two ways. First, by determining the analyte concentration outside the blood vessel, all analytes that are not capable of penetrating through the blood vessel wall 108 cannot become subject to spectroscopic analysis. Second, since the biosensing substrates 102, 104 provide selective adsorption of dedicated analytes or molecules, only those molecules become subject to spectroscopic analysis such as SERS that are suitable to be adsorbed at the biosensing substrates 102, 104.

The imaging system 114 of the spectroscopic apparatus is adapted to derive a visual image of the area in the vicinity of the blood vessel 106. Based on the derived image, the imaging system 114 is further capable of performing image processing in order to identify or to recognize the implanted biosensing substrates 102, 104. For instance, substrate detection and location determination may be performed by making use of a fluorescent marker on the surface of the biosensing substrates 102, 104. In this way contrast of a derived visual image can be appreciably enhanced with respect to the biosensing substrates 102, 104. The optical radiation required for obtaining a visual image of the region of interest in the vicinity of the blood vessel 106 is preferably coupled to the bulk of the tissue by means of the objective lens 110 and the light coupling arrangement 112. Typically, the light coupling arrangement 112 has a plurality of beam guiding and beam steering components, such as mirrors, beam splitters and in particular dichroic mirrors and dichroic beam splitters for separation of radiation, featuring different wavelengths.

Once the position of a dedicated biosensing substrate 102 has been determined by means of the imaging system 114, the output of the imaging system or the location information gathered by the imaging system 114 can be used in order to steer the objective control 120 for appropriately tuning and adjusting of the focusing arrangement, hence the objective lens 110, for focusing of excitation radiation 126 on the selected biosensing substrate 102. The excitation radiation, that is typically in the infrared or near infrared wavelength range is provided by the light source 118, that is typically implemented by means of a laser light source. Irradiation of the biosensing substrate 102 with intense excitation radiation leads to a plurality of Raman processes which lead to a shift of wavelength between incident radiation and scattered radiation that is characteristic of the analyte or molecule.

Due to the Surface-Enhancing Raman effect that leads to a substantial amplification of Raman shifted radiation, that portion of scattered radiation that stems from the Surface-Enhanced Raman effect may constitute a major part of the return radiation 128 entering the objective lens 110 and the light coupling arrangement 112 in a counter propagating way as the excitation radiation 126. Due to the wavelength shift between excitation radiation and Raman shifted signals, by means of dichroic elements of the light coupling arrangement 112, the relevant Raman shifted radiation can be selectively coupled to the spectroscopic system 116, where the Surface-Enhanced Raman signals become subject to further spectroscopic analysis in order to determine the concentration of the dedicated analyte.

FIG. 2 illustrates a cross sectional view of a biosensing substrate 102. The biosensing structure 102 features a flat substrate 130 that has numerous nano-particles 132, 134 which preferably comprise a noble metal, such as Ag or Au. The noble metal particles are prepared to selectively bind those analytes or molecules 122, 124 that will become subject to spectroscopic analysis. Preferably, the molecules or analytes 122, 124 are reversibly bound to the nano-particles 132, 134 in the sense that the number of bound molecules represents the molecule concentration around the biosensing substrate 102.

The nanosensing substrate 102 may further be adapted to selectively bind only a specific type of molecule or analyte, such as e.g. glucose. Hence, the biosensing substrate may further be provided with long-tailed thiols or mercaptans that prevent adhesion or binding of e.g. molecules or proteins that are much larger in size than the analyte of interest, e.g. glucose. In principle, EG3-modified AGFON substrates provide reversible as well as selective binding of glucose that may serve as a candidate for implantation into the bulk of the tissue in close proximity to the blood vessel 106. For further information and specifications on EG-modified AgFON substrates refer to U.S. 2004/0180379 A1 and “a glucose biosensor based on Surface-Enhanced Raman scattering: improved partition layer, temporal stability, reversibility, and resistence to serum protein interference” by Yanson, Haynes, Zhang et al, Analytical Chemistry, volume 76, number 1, 2004, page 76-85, which is herein incorporated by reference.

FIG. 3 shows a flowchart of performing the inventive method of determining analyte concentration. Here, the method of determining analyte concentration is illustrated as a two-step process but may arbitrarily split into two processes that allow to be executed at different locations. The first process describes implantation of the biosensing substrate into the biological sample. Therefore, in a first step 200 the position of the blood vessel has to be determined. This determination is preferably performed by means of the imaging system of the spectroscopic apparatus but may also be performed by means of a separate apparatus that is applicable for deriving a visual image of an area of interest around the blood vessel 106. Once the position of the blood vessel has been determined, in the following step 202 at least one substrate, typically a large number of biosensing substrates is implanted in close proximity to the blood vessel wall outside the blood vessel. This implantation may have to be performed by a skilled and trained medical personnel since it represents a minimally invasive procedure that might be harmful to the person or the animal. After step 202, hence after implantation of the biosensing substrates, the second procedure of determination of analyte concentration can be repeatedly performed as long as the implanted substrates are in well-defined proximity to the blood vessel wall and as long as the substrates are not entirely encapsulated by e.g. collagen that might degrade signal strength of the Raman signals. It is to be pointed out that implantation of the biosensing substrates can be performed by making use of the inventive spectroscopic apparatus, but it may also be performed by some other standard technique for injecting a surface-enhancing substrate into the bulk of tissue.

After implantation of the substrate in the subsequent step 204, the position of at least one implanted substrate is determined by making use of the imaging system 114 of the spectroscopic apparatus. Having identified even a plurality of biosensing substrates, the imaging system may even provide selection of one or several biosensing substrates that are suitable for Surface-Enhanced Raman Spectroscopy. Hence, by making use of the imaging system, at least a volume or a region of interest can be determined that is suitable for Surface-Enhanced Raman Spectroscopy.

Once a volume or an area of interest has been determined or once a dedicated implanted substrate has been selected by making use of the imaging system, in the following step 206, the excitation radiation is focused on the selected implanted substrate or in the area or volume of interest. In this way, the biosensing substrate and its adsorbed analytes or molecules become subject to a rather intense irradiation with excitation radiation. Due to the analyte or molecule specific binding to the biosensing substrates, an appreciable Surface-Enhanced Raman signal might be generated that can be detected in the subsequent step 208.

In step 208 radiation returning to the spectroscopic apparatus via objective lens 110 is coupled to a respective radiation detector, whose output is finally processed by means of the spectroscopic system 116. This spectral analysis of the return radiation is performed in step 210 and based on this analysis, in the final step 212 a determination of the concentration of the analyte can be performed. This determination of the concentration at least utilizes the analyzed spectrum of the return radiation but may also account for various additional parameters that might be provided by a user or that might be obtained from the imaging system 114, such as a distance parameter.

In particular, with the help of these additional parameters the analyte concentration in a blood vessel or in a vascular system can be precisely estimated based on the measured analyte concentration in the proximity of the blood vessel due to a known correlation between analyte concentration inside the blood vessel and analyte concentration outside the blood vessel. This correlation is preferably obtained by means of a calibration procedure. For instance, the calibration procedure may be performed with respect to a single individual sample, by comparing the spectroscopically derived analyte concentration with a conventionally obtained analyte concentration.

Alternatively, a calibration may be performed with respect to a large diversity of reference samples having a known or predefined analyte concentration. After termination of step 210, hence after determination of an analyte concentration, the method may return to step 204, where a subsequent spectral analysis can be performed by making use of the implanted signal enhancing substrates. In this subsequent analysis, the method is even non-invasive because a previously implanted substrate can be reused.

The invention therefore provides an improved spectroscopic apparatus and an improved method of in vivo determination of e.g. glucose concentration in blood. By performing a Surface-Enhanced Raman Spectroscopic analysis on the basis of biosensing substrates that are located in close proximity to a capillary vessel, scattering effects of red blood cells as well as interference effects of Raman signals of various constituents of the bodily fluid can be effectively reduced. In essence the acquired signals provide a higher signal-to-noise ratio and allow to reduce the overall power of the spectroscopic excitation radiation, which is beneficial to the patient health and to laser safety precautions.

### LIST OF REFERENCE NUMERALS

100 skin surface
102 substrate
104 substrate
106 blood vessel
108 blood vessel wall
110 objective lens
112 light coupling arrangement
114 imaging system
116 spectroscopic system
118 light source
120 objective control
122 analyte
124 analyte
126 excitation radiation
128 return radiation
130 substrate
132 nano-particle
134 nano-particle

1. A spectroscopic apparatus adapted to determine a concentration of an analyte (122, 124) of a biological fluid located in a volume (106) of a biological sample, the volume being confined by walls (108), which are at least semi-permeable for the analyte of the fluid, the apparatus comprising:
an imaging system (114) for determining a position of at least one substrate which is implanted into the biological sample in the proximity of the capillary vessel outside the capillary vessel, the at least one substrate being capable of inducing a signal enhancing effect,
a radiation source (118) for generating excitation radiation,
a focusing arrangement (110) for focusing of the excitation radiation on the implanted substrate,
a radiation detector for detecting return radiation emanating from the biological sample,
a spectroscopic analysis system (116) for spectrally analyzing detected return radiation and for determining the concentration of the analyte by making use of the detected return radiation.

2. The apparatus according to claim 1, wherein the focusing arrangement (110) is controllable by means of an output of the imaging system (114) for selectively focusing of the excitation radiation on the implanted substrate.

3. The apparatus according to claim 1, wherein the imaging system (114) is adapted to determine the position of the at least one implanted substrate (104) by means of a detectable mark on the at least one implanted substrate.

4. The apparatus according to claim 1, wherein the signal enhancing effect is a surface enhanced spectroscopic effect.

5. The apparatus according to claim 1, further being applicable to determine the concentration of an analyte of a biological fluid flowing through the vascular system (106) of a person, an animal or a plant and wherein the analyte (122, 124) is able to penetrate through the vessel wall (108) of the vascular system.

6. The apparatus according to claim 1, wherein the analyte (122, 124) is glucose and wherein the capillary vessel (106) is a blood vessel underneath the surface of the skin (100) of a person or an animal.

7. The apparatus according to claim 1, being further applicable to the cornea of a person or an animal and being further adapted to determine a concentration of an analyte of the aqueous humour.

8. The apparatus according to claim 1, wherein the at least one substrate (102) comprises a noble metal and wherein the substrate is further adapted to adsorb the analyte (122, 124).

9. The apparatus according to claim 1, wherein the at least one substrate is further adapted for reversible and selective adsorption of the analyte (122, 124).

10. The apparatus according to claim 1, wherein the substrate comprises noble metal nano-particles having a radius of curvature in the range of nanometers.

11. A method of determining a concentration of an analyte (122, 124) of a biological fluid located in a volume (106) of a biological sample, the volume being confined by walls (108), which are at least semi-permeable for the analyte of the fluid, the method comprising the steps of:
determining a position of at least one substrate (104) which is implanted into the biological sample in the proximity of the capillary vessel outside the capillary vessel, the at least one substrate being capable of inducing a signal enhancing effect,
focusing excitation radiation on the implanted substrate and detecting return radiation,
spectrally analyzing the detected return radiation for determining the concentration of the analyte.

12. The method according to claim 11, further being applicable to in vivo determination of the concentration of an analyte of the biological fluid flowing through the vascular system (106) of a person, an animal or a plant and wherein the analyte (122, 124) is able to penetrate through the vessel walls (108) of the vascular system.

13. The method according to claim 11, further being applicable to the cornea of a person or an animal and being further adapted to determine a concentration of an analyte of the tear liquid and/or aqueous humour.

14. A computer program product for a spectroscopic apparatus adapted to determine a concentration of an analyte (122, 124) of a fluid, which is located in a volume (106) of a biological sample, the volume being confined by walls (108) which are at least semi-permeable for the analyte of the fluid, the computer program product comprising computer program means which can be operated:
to process an output signal of an imaging system (114) to determine a position of at least one substrate (104), which is implanted into the biological sample in the proximity of the capillary vessel (106) outside the capillary vessel, the at least one substrate being capable of inducing a signal-enhancing effect,
to control a focusing arrangement for focusing an excitation radiation on the implanted substrate,
to process an output signal of a radiation detector for spectral analysis of the detected return radiation to determine the concentration of the analyte, the output signal being indicative of return radiation detected by means of the radiation detector.
